(19) United States
(12) Patent Application Publication
(10) Pub. No.: US 2004/0230183 A1
(43) Pub. Date: Nov. 18, 2004

(54) Drug delivery device and syringe for filling the same

(76) Inventors: Wisam Breegi, Woburn, MA (US); Pericles Calias, Melrose, MA (US); Anthony P. Adamis, Boston, MA (US); David Shima, Boston, MA (US); Emmett T. Cunningham, New York, NY (US)

Correspondence Address: EYETECH PHARMACEUTICALS, INC., 3 TIMES SQUARE 12TH FLOOR, NEW YORK, NY, 10036, US

(21) Appl. No.: 10/781,350

(22) Filed: Feb. 18, 2004

Related U.S. Application Data
(60) Provisional application No. 60/447,971, filed on Feb. 18, 2003.

(51) Int. Cl. A61K 9/22

(52) U.S. Cl. 604/891.1

(57) ABSTRACT

The invention relates to the field of drug delivery devices. More particularly, the invention relates to implantable, refillable drug delivery devices which provide for drug delivery over sustained time periods. The present invention has particular application for ophthalmic drug delivery applications.

### RELATED APPLICATIONS

This application claims priority to U.S. Provisional Application Ser. No. 60/447,971, filed Feb. 18, 2003, which application is hereby incorporated in its entirety by reference.

### FIELD OF THE INVENTION

The invention relates to the field of drug delivery devices. More particularly, the invention relates to implantable, refillable drug delivery devices which provide for drug delivery over sustained time periods. The present invention has particular application for ophthalmic drug delivery applications.

### BACKGROUND OF THE INVENTION

The efficacy of a medical treatment using drugs often requires controlled delivery of the drug to a particular location. In certain therapies, it is necessary to repeatedly administer the drug over a long period of time, especially when the drug is rapidly degraded or flushed from the area requiring treatment, e.g., the eye. Drug delivery in such therapies is problematic. The delivery of drugs to the eyes is particularly challenging because the interior regions of the eye are difficult to medicate through systemically administered drugs and topically administered drugs are usually administered at elevated, and potentially dangerous concentrations, because the production of tears flushes the majority of the drug from the eye in a short time. In addition to physiological barriers to drug delivery, patients often forget to take their medication, or they are unwilling or unable to administer the drug. Localized drug delivery may also be necessary when a drug is inappropriate for systemic delivery.

Several methods exist for localized delivery of drugs. These methods include the use of topically applied formulations (e.g., patches and viscous gels), controlled release formulations, and drug delivery devices. While the use of topically applied formulations allows for ease of administration, any localized effect is limited to an area that is easily accessible, and for treatment of the eyes, the use of gels may impair the vision of the patient. Controlled release formulations provide drug release over a period of time, for example, by diffusion of the drug out of the polymer and/or degradation of the polymer, but it is also difficult to retain such formulations in a particular area. Mechanical drug delivery devices have also been developed for localized delivery and have the advantage of being able to physically separate a drug from the body; however, many such devices are mechanically complex and difficult or impossible to refill. Thus, there is a need for new drug delivery devices.

### SUMMARY OF THE INVENTION

The invention features a drug delivery device for delivery of therapeutic compounds, a syringe for filling the device, and methods of use thereof. The invention is based on a design ensuring emptying or refilling of the device without creating large pressure changes within the device. According to one embodiment, an expandable chamber within a hard, protective shell contains a drug that is allowed to contact the desired treatment site at a controlled rate. The device is designed such that it can be emptied and refilled without creating large pressure changes in the device that could cause the device to detach from the treatment site or injure the surrounding tissue.

In one aspect, the invention relates to ophthalmic drug delivery devices and features a device for transscleral delivery of a therapeutic agent to the eye of a mammal. The device includes a) a dome; b) a membrane disposed within the dome, wherein the membrane divides the interior of the dome into a pressure equalizing chamber and a therapeutic agent chamber, wherein the volume of the pressure equalizing chamber changes in response to the volume in the therapeutic agent chamber in order to maintain a substantially constant pressure within the dome over a period of at least one month; and c) a rim connected to the membrane and adapted to be affixed to the sclera of the eye (e.g., throughout the circumference of the rim) that, when so affixed, defines a region of the eye which is in fluid communication with the therapeutic agent chamber.

In another aspect, the device also includes a port through which a therapeutic agent can be injected into the therapeutic agent chamber. Such a port includes, for example, first and second septa, wherein the first septum separates the exterior of the device from the pressure equalizing chamber, and wherein the second septum separates the pressure equalizing chamber from the therapeutic agent chamber. The first and second septa may be disposed such that a needle is capable of piercing both septa simultaneously.

The invention further features a syringe for injecting fluid into or withdrawing fluid from a closed system including a) a barrel having a fluid portal end and a pressure generating end; b) a needle having a hollow bore and being connected to the fluid portal end; c) a venting tube having a hollow bore and being connected to the needle, wherein the hollow bores of the venting tube and the needle are not in fluid communication; and d) a pressure source (e.g., a plunger) connected to the pressure generating end of the barrel.

In another aspect, the invention features a method of injecting fluid into or withdrawing fluid from a closed system including the steps of a) providing a syringe of the invention; b) passing the needle and venting tube through a port into the system; and c) injecting fluid into or withdrawing fluid from the system, wherein when fluid from the barrel is injected into the system through the needle, fluid inside the system exits through the venting tube in order to maintain a substantially constant pressure within the system, and when fluid from the system is pulled into the barrel through the needle, fluid outside the system enters the system through the venting tube in order to maintain a substantially constant pressure within the system. The above-described methods may be used to deliver a therapeutic agent to a closed system, such as the therapeutic agent chamber of a device of the invention.

The invention also features a method of treating an ocular disease state in a mammal. Such methods include the steps of a) affixing to the sclera of the eye of the mammal the rim of a device of the invention, and b) loading the device with a therapeutic agent that treats the disease state. The method may be used to deliver a therapeutic agent to the choroid or retina.

Other features and advantages of the invention will be apparent from the following description, the drawings, and the claims.

### BRIEF DESCRIPTION OF THE DRAWINGS

FIG. 1A is a cross-sectional view of a device of the invention.

FIG. 1B is a cross-sectional view of an injection port of a device of the invention.

FIG. 1C is an edge view of a device of the invention.

FIG. 1D is a view from the base of a device of the invention.

FIGS. 2A and 2B are exploded views of the dome and the rim of the device.

FIG. 3A is a view from the base of a device of the present invention.

FIG. 3B is a cross-sectional view of a device of the invention.

FIG. 3C is a view from the base of a device of the present invention.

FIG. 4A is a schematic view of the syringe of the invention.

FIG. 4B is a schematic view of the syringe of invention in the injection port of the invention.

FIG. 5 is a schematic representation of the structure of pegaptanib sodium.

### DEFINITION OF TERMS

By an “aptamer” is meant a nucleic acid that binds to another chemical species, or portion thereof, by any means other than hybridization.

By a “closed system” is meant a container that becomes pressurized, at least temporarily, when a fluid in introduced into it. Closed systems may contain openings that are capable of releasing internal pressure over time.

By two volumes being “in fluid communication” is meant two volumes connected such that liquid or gas can pass between the two.

By “substantially constant pressure” is meant pressure that is constant with minor, temporary variations due to filling, emptying, or a change in osmotic pressure of the surrounding liquid.

By “therapeutic agent” is meant any compound or mixture of compounds that provide a therapeutic effect for one or more diseases, disorders, or conditions. Such compounds include, without limitation, small organic or inorganic molecules, proteins (e.g., antibodies), peptides, lipids (e.g., steroids) and nucleic acids (e.g., aptamers). Therapeutic agents are, for example, antibiotics, analgesics, antiinflammatory compounds, or any other compound for the treatment of a disease, disorder, or condition.

By “treating” is meant the medical management of a patient with the intent that a cure, amelioration, or prevention of a disease, pathological condition, or disorder will result. This term includes active treatment, that is, treatment directed specifically toward improvement of a disease, pathological condition, or disorder, and also includes causal treatment, that is, treatment directed toward removal of the cause of the disease, pathological condition, or disorder. In addition, this term includes palliative treatment, that is, treatment designed for the relief of symptoms rather than the curing of the disease, pathological condition, or disorder; preventive treatment, that is, treatment directed to prevention of the disease, pathological condition, or disorder; and supportive treatment, that is, treatment employed to supplement another specific therapy directed toward the improvement of the disease, pathological condition, or disorder. The term “treating” also includes symptomatic treatment, that is, treatment directed toward constitutional symptoms of the disease, pathological condition, or disorder.

### DETAILED DESCRIPTION OF THE INVENTION

The invention features a drug delivery device that is attached to an internal body surface in order to deliver a therapeutic compound and drug delivery methods related thereto. Once attached to the internal body surface, the device of the present invention is refillable without large pressure changes within the device during emptying or refilling. The invention also features a syringe for filling the device and methods of using the device to treat a disease, disorder, or condition.

The invention in one aspect features a drug delivery device that is attached to the eye and used for ophthalmic drug delivery applications. According to this aspect of the invention, the device of the invention allows for the controlled localized delivery of a therapeutic agent to the eye. Because it is attached directly to the sclera, the device can be used to administer drugs to the interior tissues of the eye. In addition, the design of the device allows for emptying and refilling with minimal pressure change in the device in order to prevent detachment of the device or injury to the eye. The syringe of the invention also allows emptying, refilling, and pressure equalization with one needle stab, which minimizes the time and complexity of maintaining and using the device.

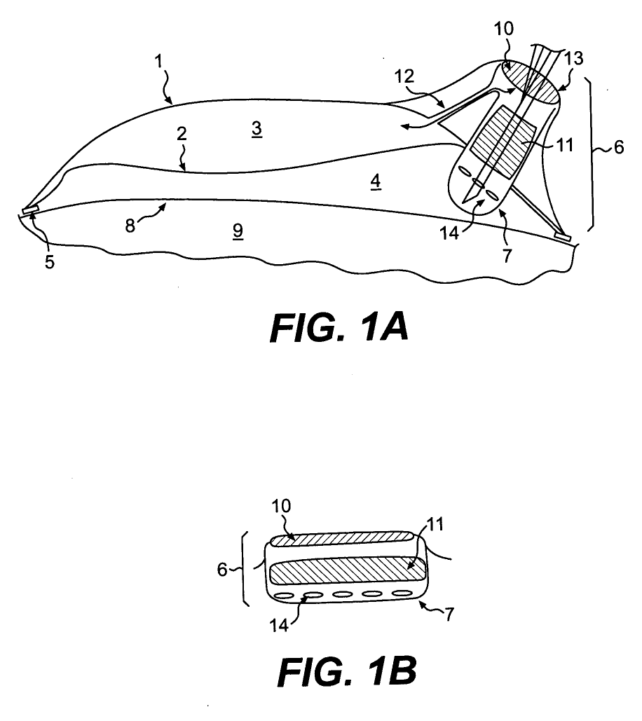

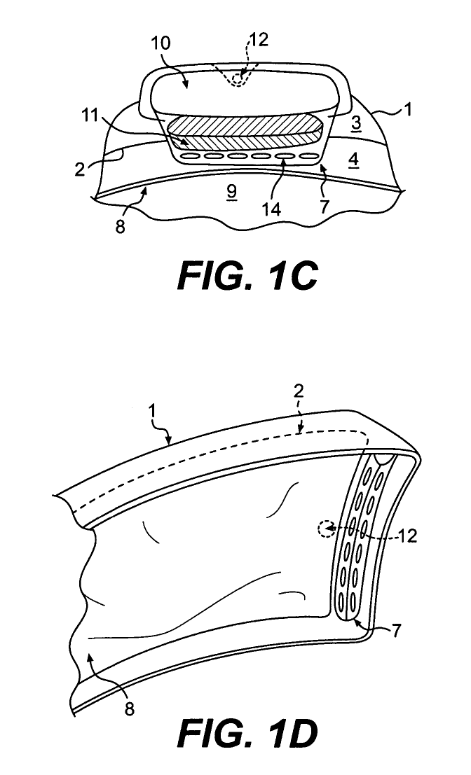

Referring to FIG. 1A, the device includes an outer layer or dome 1 and an inner membrane 2. The membrane 2 divides the interior of the dome into a pressure equalizing chamber 3 and a therapeutic agent chamber 4. The volume of the therapeutic agent chamber 4 may be formed to accommodate from about 0.001 ml to about 250 ml. The size of the chamber is determined, in part, by the volume of the dose to be administered. For ophthalmic uses, the therapeutic agent chamber 4 has a volume of, for example, 1 μl to 1 ml. In addition, the device includes a rim 5 that is affixed to the desired site of location, such as the sclera 9. The device also includes an injection port 6 for fluid transfer to and from the therapeutic agent chamber 4 and the pressure equalizing chamber 3. The device may also include a puncture guard 7 inside the therapeutic agent chamber 4 in order to prevent a needle, or other conduit for introduction or removal of fluid, inserted in the therapeutic agent chamber 4 from injuring the eye or surrounding tissue.

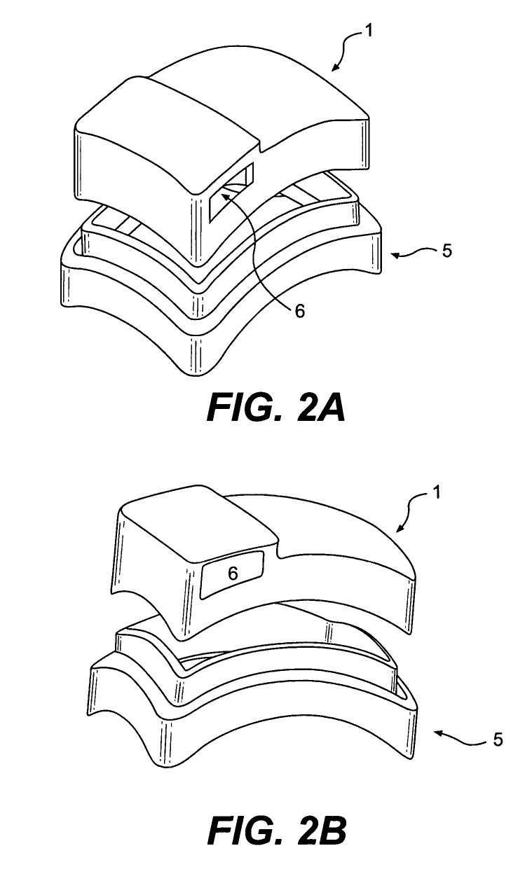

In one embodiment, the therapeutic agent chamber 4 also includes a base 8 in fluid communication with the rim 5. Exemplary dimensions for the device are 16.5×12.5×4.2 mm (l×w×h) (FIG. 2). The dimensions of the device will depend on the area of the eye to which it is attached, the materials used in its construction, and the amount of therapeutic agent to be delivered.

The dome 1 (FIG. 2A and 2B) is composed of a hard material, such as metal (e.g., titanium, nickel, nitinol, gold, stainless steel, tantalum), carbon, polyethylene, polypropylene, polycarbonate, polyester, epoxy, polystyrene, or urethane. The material of the dome is desirably biocompatible and non-bioerodible. Suitable materials include PEKK® (polyetherketoneketone) and PEEK® (polyetheretherketone). The dome can be manufactured by any standard method known in the art including, without limitation, machining, injection molding, stereolithography, or casting.

The membrane 2 is typically constructed from a flexible material, such as silicone, polyvinyl alcohol, ethylene vinyl acetate, polylactic acid, nylon, polypropylene, polycarbonate, cellulose, cellulose acetate, polyglycolic acid, polylactic glycolic acid, a cellulose ester, polyethersulfone, an acrylic, polytetrafluoroethylene, polyfluorinated ethylenepropylenesilastic, Dacron, Mylar, ionic salts of alginate, polycaprolactone, urethanes, polyethylene, polymethylmethacrylate, a polyester, and mixtures thereof. In one embodiment, the membrane is not taut when filled with fluid, i.e., it exerts minimal or no pressure on the contents of the therapeutic agent chamber. The membrane may be attached to the dome by any suitable means, e.g., clamping, adhesives, or a thermal weld. When attached, the membrane prevents liquid flow between the therapeutic agent and pressure equalizing chambers. The membrane may also be coated with a polymeric layer to minimize water vapor penetration into the upper chamber if necessary. The exact method of attachment will depend on the materials used in the dome and the membrane. The membrane may also be connected to the rim of the device, e.g., by being molded as one piece, and the dome may fit around the combined rim and membrane.

The injection port 6 may include a septum, a valve, or both (FIG. 1A-1C). These septa or valves are attached to the device by standard means. Desirably, the injection port includes two septa or valves, one between the pressure equalizing chamber and the exterior of the device 10 and one between the pressure equalizing chamber and the therapeutic agent chamber 11. Exemplary resealable materials for septa include silicone, polyvinyl alcohol, ethylene vinyl acetate, cellulose, cellulose acetate, a cellulose ester, polyethersulfone, polytetrafluoroethylene, polyfluorinated ethylenepropylenesilastic, Dacron, Mylar, polycaprolactone, urethanes, polyethylene, polymethylmethacrylate, a polyester, and mixtures thereof. Simple valves (e.g., mechanically, thermally, chemically, electrically or magnetically actuated) may also be used to control fluid flow into and out of the chambers of the device. Such valves are known in the art. When two septa or valves are employed (FIG. 1B and C), they are typically arranged such that a single needle or other conduit can pass through both simultaneously. In this configuration, a fluid vent 12 may be present between the pressure equalizing chamber and the volume between the two septa or valves. The exterior portion of the injection port may be colored differently from the dome in order to provide a target area 13 to aid in fluid delivery. Exemplary dimensions for the target area are approximately 4×3 mm.

The puncture guard 7 is also made from a hard material like the dome, and it can be manufactured in one piece with the dome. The puncture guard 7 is connected to the injection port and contains holes 14 that allow fluid to flow between the therapeutic agent chamber and the interior of the puncture guard. The puncture guard is designed to prevent piercing by a needle or other inserted fluid conduit. In one embodiment, the holes 14 in the puncture guard are smaller than a needle or other conduit. The holes 14 may also be arranged so that a needle cannot pass through.

The rim 5 of the device is typically curved in order to conform to the shape of the site of administration, such as the eye (FIG. 1A-1D). Exemplary materials for the rim 5 include silicone, urethane, other soft biocompatible polymers as described herein, and metals, such as titanium, tantalum, gold, nickel, nitinol, and stainless steel. The rim may be directly connected to the therapeutic agent chamber or it may be connected to the intended delivery site through a tube or catheter. Such a catheter can be manufacture from any of the materials described herein.

The rim 5 may be affixed by suturing, gluing, or sealing by means of one or more polymerizable compounds. Alternatively, the affixing includes utilizing biological healing mechanisms, such as postoperative adhesions, fibrotic encapsulation, or other foreign body reactions. The rim 5 is, for example, affixed to the sclera over the equator of the eye or over the pars planar of the eye. When a device including a catheter for localized delivery from the therapeutic chamber is employed, the rim 5 may be affixed to the sclera over the equator of the eye and the catheter may be affixed to the pars planar.

The base 8 to the therapeutic agent chamber 4 may be constructed, for example, of any of the soft or hard biocompatible materials discussed for the dome, membrane, or rim. The base provides a barrier that restricts flow between the therapeutic agent chamber and the rim. For example, the base may be a molecular weight cut-off membrane, or it may be perforated in order to allow passage of the therapeutic agent (e.g., 25% of the surface of the base may be perforations). The surface of the eye or other tissue intended for localized delivery may also serve as a barrier to prevent liquid in the therapeutic agent chamber from leaking out. The base is connected to the membrane or rim through any suitable adhesive or attachment technique. The device may further include a therapeutic agent disposed in the therapeutic agent chamber.

The therapeutic agent chamber 4 may be open to the site of administration, such as the sclera, or may include a rate controlling membrane (not shown) positioned between the therapeutic agent chamber 4 and site of administration. When a rate controlling membrane is used, the therapeutic agent chamber 4 is defined by the membrane 2 and rate controlling membrane. Suitable rate controlling membranes in whole or in part are permeable to the therapeutic agent to be administered. The rate of release of the therapeutic agent from the rate controlling membrane is controlled by a number of factors including porosity, the area of the agent permeable portion of the rate controlling membrane, the thickness of the membrane, the diffusion coefficient of the agent, particle size of the agent, concentration gradient of the agent in the therapeutic agent chamber relative to the concentration outside of the chamber, and solubility of the agent in the rate controlling membrane.

The drug-permeable portion of the rate controlling membrane can be microporous or dense without pores. Dense membranes can transport drug molecules by a solution diffusion mechanism, which is well described in the literature. See, e.g., “Controlled Release of Biologically Active Agents,” (1987) R. Baker, John Wiley & Sons. The drug-permeable portion may be in any shape, such as circular, square, rectangular, or may be irregular in shape. The rate controlling membrane may comprise more than one drug-permeable portion, which may be arranged in any manner (e.g., in rows, or in a random arrangement).

The drug-permeable portion of the rate controlling membrane may comprise an organic or synthetic polymer, including, but not limited to, polypropylene, polytetrafluoroethylene, polycarbonates, polyvinylchloride, cellulose acetate, cellulose nitrate, and polyacrylonitrile. Other suitable materials include, without limitation, polycarbonates i.e., linear polyesters of carbonic acids in which carbonate groups recur in the polymer chain by phosgenation of a dihydroxy aromatic such as bisphenol A, polyvinylchlorides, polyamides such as polyhexamethylene adipamide and other such polyamides commonly known as “nylon”, modacrylic copolymers such as those formed of polyvinylchloride and acrylonitrile, and styrene-acrylic acid copolymers, polysulfones such as those characterized by diphenylene sulfone groups in the linear chain thereof, halogenated polymers such as polyvinylidene fluoride and polyvinylfluoride, polychloroethers and thermoplastic polyethers, acetal polymers such as polyflannaldehyde, acrylic resins such as polyacrylonitrile, polymethyl methacrylate and poly n-butyl methacrylate, polyurethanes, polyimides, polybenzinlidazoles, polyvinyl acetate, aromatic and aliphatic polyethers, cellulose esters such as cellulose triacetate, cellulose, collodion, epoxy resins, olefins such as polyethylene and polypropylene, porous rubber, cross-linked poly(ethylene oxide), cross-linked polyvinylpyrrolidone, cross-linked poly(vinyl alcohol); derivatives of polystyrene such as poly (sodium styrenesulfonate) and polyvinylbenzyltdmethyl-ammonium chloride, poly(hydroxyethyl methacrylate), poly(isobutyl vinyl ether), polyisoprenes, polyalkenes, ethylene vinyl acetate copolymers such as those described in U.S. Pat. No. 4,144,317, incorporated herein by reference, polyamides, polyurethanes, polyethylene oxides, polyox, polyox blended with polyacrylic acid or Carbopol™, cellulose derivatives such as hydroxypropyl methyl cellulose, hydroxyethyl cellulose, hydroxypropyl cellulose, pectin, starch, guar gum, locust bean gum, and the like, along with blends thereof.

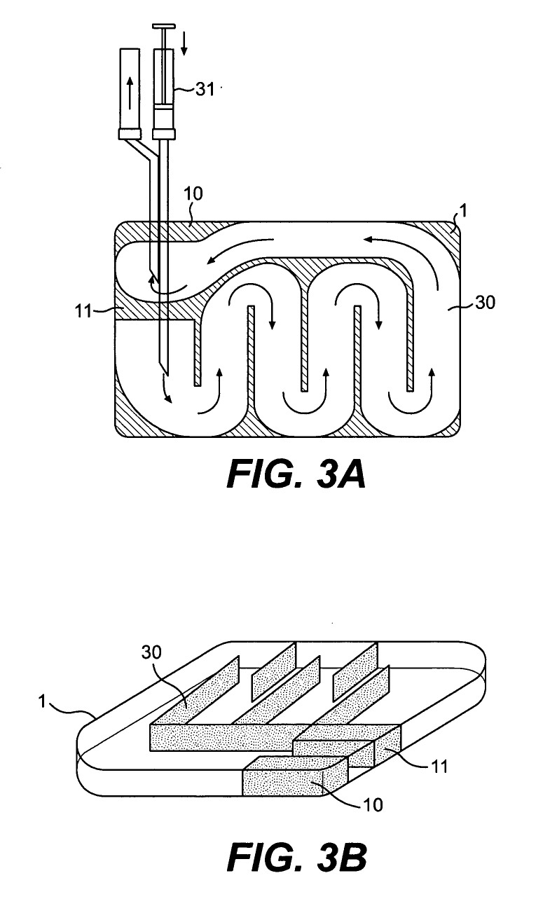

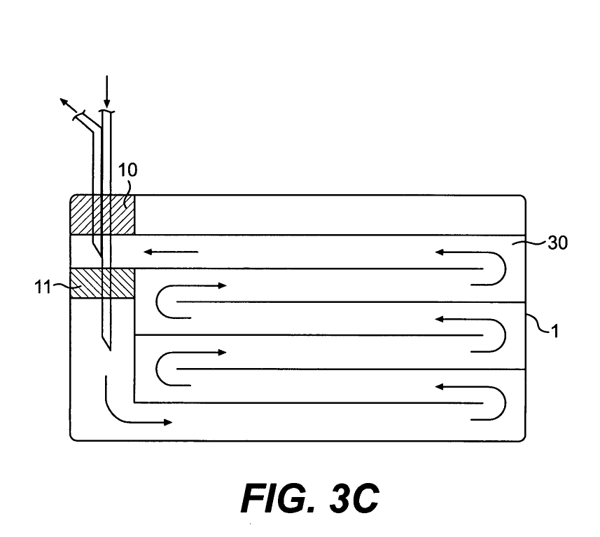

It is to be understood that the substantially isobaric conditions within the device can be maintained by various designs as alternatives to use of membrane 2. FIGS. 3 A-C depict alternative embodiments wherein a channel 30 is provided to ensure the substantially isobaric conditions within the device. The device consists of a hard outer shell 1 in which the underside is patterned to give a channel 30 and septums 10 and 11 connecting the air exchange tube of the syringe 31 with the therapeutic tube of the syringe 31. This channel may be configured in a sinusoidal manner to maximize the length of the channel 30. The base or bottom of the pathway 30 may consist of an impermeable membrane with a catheter for remote delivery. The base may also be composed of a rate controlling membrane as set forth above to attenuate the delivery of therapeutics. The base may fully or partially cover the surface area of the contact surface of the device. If the base only partially covers the contact surface, the therapeutic would have direct contact with the tissue of interest. The size of the base opening could be selected by one of ordinary skill in order to provide a predetermined delivery rate. The walls of the channel 30 will either contact the base or the tissue. The capacity or volume of therapeutic that the device will hold will depend on the height of the device as well as the thickness of the channel 30. Designs incorporating such a channel 30 contain no moving parts, will have a lower aspect ratio, and a smaller foot print.

While specific reference has been made to the use of the devices of the present invention to administer therapeutic agents to the eye, it is to be understood that the present invention can be used to deliver a therapeutic agent to any desired site, including, but not limited to, intraorbital, intraocular, intraaural, intratympanic, intrathecal, intracavitary, peritumoral, intratumoral, intraspinal, epidural, intracranial, and intracardial. As discussed above, the agent can be administered through the contact surface of the device itself, or through the use of a catheter to access the intended site of administration. Examples of catheters suitable for use with the present invention are known in the art such as disclosed in U.S. Pat. Nos. 4,692,147, 5,713,847, and 5,711,316 and in PCT applications WO 00/53253, WO 01/43528, and WO 02/24159, herein incorporated in their entirety by reference.

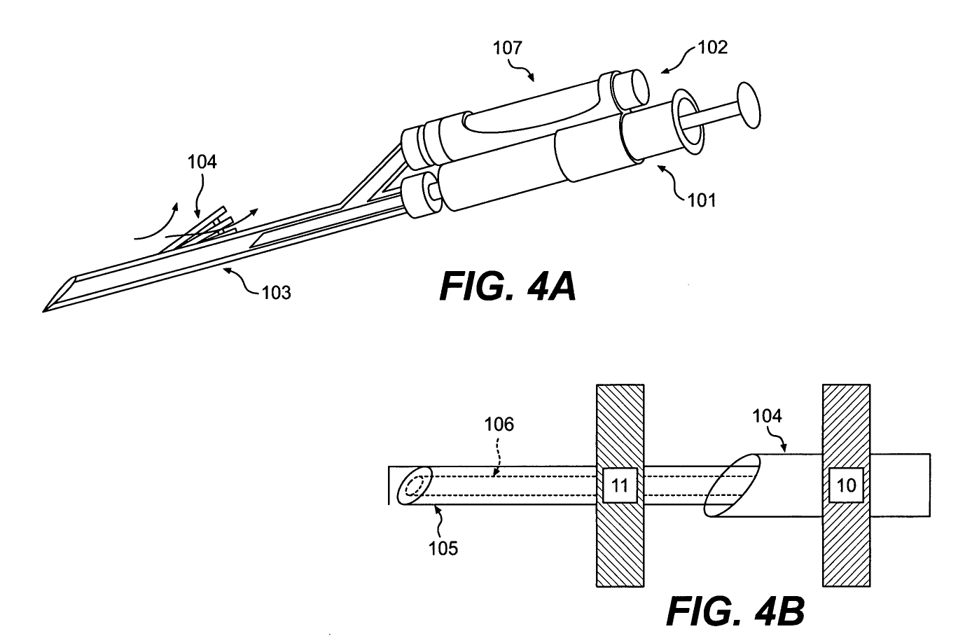

FIG. 4A shows a syringe for transferring fluid into and out of a closed system. The discussion of the syringe will focus on the device of the system, but the general principles are applicable to other closed systems including those containing only a single interior chamber. The syringe includes a barrel 101, a vacuum source 102, one or more needles 103, and a venting tube 104 for the introduction and removal of fluid to and from the interior of the device as described herein. The barrel contains the therapeutic agent dissolved or suspended in a liquid formulation. The needles allow for removal of fluid from the therapeutic agent chamber and introduction of fluid into the therapeutic agent chamber, and the venting tube allows flow of fluid into and out of the pressure equalizing chamber. A single needle with two or more hollow bores may perform all three functions or multiple needles/tubes may be used (FIG. 4B). When one needle is employed, the hollow bores may be arranged in a concentric pattern. In such a concentric pattern, the venting tube is typically the outermost bore, and shorter than the other two bores. Of the two inner bores, one 105 is for introduction of fluid into the therapeutic agent chamber, and one 106 is for removal of fluid from the therapeutic agent chamber. Using such a configuration, the venting tube does not extend into the therapeutic agent chamber when the other two bores do. In another embodiment, the venting tube is attached to the exterior of a needle. This venting tube may be hinged such that it extends away from the needle when it is inserted into the device (FIG. 1A and 4A). Needles/tubes may be manufactured from standard materials, e.g., stainless steel, by methods known in the art.

The vacuum source 102 may be any source of low pressure, such as a plunger that increases the volume of a chamber, a vacuum pump (e.g., an aspirator), or an evacuated canister. The use of an evacuated canister that can be removed from the syringe and sealed allows for the collection of fluid within the therapeutic agent chamber, which can be examined for microbial growth or tested for the presence of markers for beneficial or detrimental effects. The canister may also contain a window 107 for visual inspection of the contents.

In one embodiment, an evacuated canister can be provided with an indicator to show that the canister is in fluid contact with the therapeutic agent chamber 14 for aspirating the contents thereof prior to filling chamber 14 with a therapeutic agent formulation. According to this embodiment, a flexible diaphragm membrane is provided within the canister. The diaphragm is visible through window 107 such that when the evacuated canister is actuated to aspirate contents of therapeutic agent chamber 14, the diaphragm flexes in a direction away from the needle. Such movement of the diaphragm or a sliding gauge confirms to the user that a vacuum is being drawn within the therapeutic agent chamber 14. Thus, this provides a visual indication to the user that the device is functioning properly. In addition to a flexible diaphragm, other designs for the indicator are readily apparent to one of ordinary skill including a spring resistant gauge, septum, or use of a pliable plastic.

The syringe may further include a vacuum source in fluid communication with the needle or a second needle having a hollow bore that is not in fluid communication with the hollow bore of the needle and a vacuum source in fluid communication with the hollow bore of the second needle. The vacuum source is, for example, an evacuated canister that may include a window for visual inspection of the contents. The evacuated canister may also be removable and sealable. The hollow bores of all or any subset of the needles/tubes may be coaxial to one another.

The syringe is used to empty and/or fill a closed system. The closed system is, for example, a device of the invention, wherein when fluid from the barrel of the syringe is injected into the therapeutic agent chamber through the needle, fluid inside the pressure equalizing chamber exits through the venting tube in order to maintain a substantially constant pressure within the device, and when fluid from the therapeutic agent chamber is pulled into the barrel through the needle, fluid outside the device enters the pressure equalizing chamber through the venting tube in order to maintain a substantially constant pressure within the device.

The invention further features a method of injecting fluid into or withdrawing fluid from a closed system including the steps of a) providing a syringe of the invention having two needles as described above; b) passing both needles and the venting tube through a port into the system; and c) injecting fluid into or withdrawing fluid from the system; wherein when fluid from the barrel is injected into the system through the needle, fluid inside the system exits through the venting tube in order to maintain a substantially constant pressure within the system, and when fluid in the system is pulled into the vacuum source through the second needle, fluid from outside the system enters the system through the venting tube in order to maintain a substantially constant pressure within the system. The closed system is, for example, a device of the invention, wherein when fluid from the barrel is injected into the therapeutic agent chamber through the needle, fluid inside the pressure equalizing chamber exits through the venting tube in order to maintain a substantially constant pressure within the device, and when fluid in the therapeutic agent chamber is pulled into the vacuum source through the second needle, fluid from outside the device enters the pressure equalizing chamber through the venting tube in order to maintain a substantially constant pressure within the device. Actuating the vacuum source may be used to remove fluid from the therapeutic agent chamber while increasing the pressure in the barrel may be used to inject fluid into the therapeutic agent chamber.

For ophthalmic applications, the device is affixed to the sciera by the rim. The attachment may be made by sutures or by biocompatible adhesives, e.g., polymerizable compounds or biological adhesives from postoperative adhesions, fibrotic encapsulation, or other foreign body reactions. In certain embodiments, the outer layers (e.g., the Tenon's capsule) of the eye may be removed or scored prior to attachment in order to enable drug delivery into the interior of the eye, e.g., the vitreous humor. Alternatively, the sclera may be thinned prior to attachment. The attachment of the rim to the sclera provides a liquid tight seal, and once attached, the surface of the eye is in fluid communication with the therapeutic agent chamber. The circumference of the rim determines the area of therapeutic delivery. If present, a rate controlling membrane regulates the rate of contact of a therapeutic agent with the eye. The tissue at the site of administration and/or the rate controlling membrane determine the rate at which a particular drug is delivered. The rim is attached to the sclera, for example, over the equator or pars planar of the eye. The rim may, for example, be attached to the sclera throughout the circumference of the rim.

The mechanism of emptying and filling the therapeutic agent chamber using the syringe of the invention and an injection port including two septa is as follows. The syringe needle pierces the outer septum on the injection port and enters a chamber that is directly connected to the pressure equalizing chamber. The needle then passes through the second septa and is stopped by the puncture guard. As the needle is stopped, the venting tube is inserted into the first septum providing a channel for fluid (typically air) to flow into and out of the pressure equalizing chamber (FIG. 1A and 4B). For the initial filling, liquid containing a therapeutic agent is injected into the therapeutic agent chamber from the barrel of the syringe and through the holes in the puncture guard. As the liquid enters the therapeutic agent chamber, the chamber expands by forcing the membrane into the pressure equalizing chamber. Air in the pressure equalizing chamber then exits through the venting tube in order to maintain a substantially constant pressure within the device.

For subsequent fillings, any fluid in the therapeutic agent chamber is first removed by actuating the vacuum source (typically an evacuated canister) causing the pressure equalizing chamber to expand as the membrane collapses. Air then enters the pressure equalizing chamber to maintain a substantially constant pressure within the device. Any liquid or particulate matter removed from the therapeutic agent chamber can be stored in an evacuated canister for later visual inspection or laboratory testing. After the therapeutic chamber is emptied, it can be filled as previously described. Once the needle is removed, the septa reseal to maintain fluid tight chambers. It is envisioned that the refilling process can occur within 1 min, e.g., 30 seconds, depending on the amount of fluid to be delivered.

When a syringe with one needle having one bore is employed, some fluid removed from the therapeutic agent chamber may remain in the needle bore and be re-injected with new fluid. When a syringe with multiple needles or a needle having more than one bore is employed, fluid removed passes through one bore to the vacuum source, while fluid introduced passes from the barrel through another bore into the therapeutic agent chamber. In this configuration, fluid removed from the chamber is not re-injected.

Although the above process was described using the syringe of the invention, alternative methods of filling can be employed. For example, a needle can be inserted to serve as a venting tube; one syringe can be employed to remove fluid from the therapeutic agent chamber; and another syringe can be employed to inject fluid. In addition, valves or a combination of a valve and a septum may be used in place of two septa. For example, a needle pierces a septum in order to deliver or remove fluid to the therapeutic agent chamber, while a valve is opened to allow the flow of air into and out of the pressure equalizing chamber. Other variations on these configurations will be apparent to one skilled in the art.

The above-described methods for filling the device of the invention are designed to minimize any changes in pressure in the device when it is being filled or emptied. One skilled in the art will recognize that the pressure will not necessarily be constant during the actual introduction or removal of fluid since the introduction or removal may occur at a faster rate than air can pass through the venting tube to equalize the pressure. The venting tube should thus be maintained in the device until the internal pressure has equalized. Careful control of the rate of introduction or removal of fluid will minimize any temporary changes in pressure in the device.

A device of the invention may be used in the treatment of any eye disease. A device of the invention may also be used to direct a therapeutic agent to a particular eye tissue, e.g., the retina or the choroid. The therapeutic agent or combination of agents will be chosen based on the disease, disorder, or condition being treated. In addition to a therapeutic agent for a particular condition, other compounds may be included for secondary effects, for example, an antibiotic to prevent microbial growth. The amount and frequency of the dosage will depend on the disease, disorder, or condition being treated and the therapeutic agent employed. One skilled in the art can make this determination.

Therapeutic agents that may be employed in the device of the invention include, without limitation, small molecules, hormones, proteins, peptides, aptamers, antibodies, lipids, glycolipids, DNA, RNA, PNA, enzymes, sugars, saccharides, glycoproteins, polymers, metalloproteases, transition metals, or chelators. In addition, nucleic acid vectors can also be delivered wherein the nucleic acid may be expressed to produce a protein that may have a variety of pharmacological, physiological or immunological activities. Macromolecules with a molecular weight of about 5 KD to about 500 KD may also be used in accordance with the invention.

For ophthalmic drug delivery applications, exemplary disease states include macular degeneration, diabetic retinopathy, glaucoma, optic disc neovascularization, iris neovascularization, retinal neovascularization, choroidal neovascularization, pannus, pterygium, macular edema, vascular retinopathy, retinal vein occlusion, histoplasmosis, ischemic retinal disease, retinal degeneration, uveitis, inflammatory diseases of the retina, keratitis, cytomegalovirus retinitis, an infection, conjunctivitis, cystoid macular edema, cancer, and proliferative vitreoretinopathy.

Classes of therapeutic agents include anti-infectives including, without limitation, antibiotics, antivirals, and antifungals; analgesics; antiallergenic agents; mast cell stabilizers; steroidal and non-steroidal anti-inflammatory agents; decongestants; anti-glaucoma agents including, without limitation, adrenergics, beta-adrenergic blocking agents, alpha-adrenergic blocking agonists, parasympathomimetic agents, cholinesterase inhibitors, carbonic anhydrase inhibitors, and protaglandins; antioxidants; nutritional supplements; angiogenesis inhibitors; antimetabolites; fibrinolytics; wound modulating agents; neuroprotective drugs; angiostatic steroids; mydriatics; cyclopegic mydriatics; miotics; vasoconstrictors; vasodilators; anticlotting agents; anticancer agents; immunomodulatory agents; VEGF antagonists; immunosuppresant agents; and combinations and prodrugs thereof.

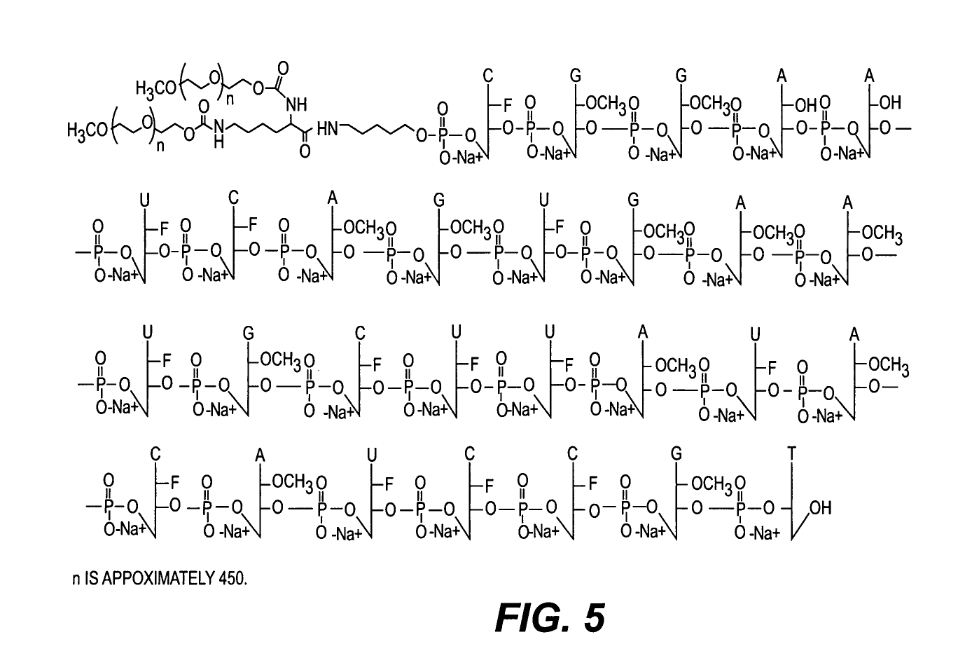

Specific therapeutic agents include pegaptanib sodium (EYE001 or NX31838 as described in U.S. Pat. No. 6,051,698, herein incorporated in its entirety by reference, and seen in FIG. 5), 4,9(11)-pregnadien-17α,21-diol-3,20-dione, 4,9(11)-pregnadien-17α,21-diol-3,20-dione-21-acetate, timolol, betaxolol, atenolol, brimonidine, acetazolamide, methazolamide, dichlorphenamide, diamox, nimodipine, eliprodil, colchicine, vincristine, cytochalasin B, tetracycline, chlortetracycline, bacitracin, neomycin, polymyxin, gramicidin, oxytetracycline, chloramphenicol, gentamycin, erythromycin, sulfonamides, sulfacetamide, sulfamethizole, sulfisoxazole, fluconazole, nitrofurazone, amphotericin B, ketoconazole, trifluorothymidine, acyclovir, ganciclovir, didanosine, AZT, foscamet, vidarabine, idoxuridine, ribavirin, protease inhibitors, anti-cytomegalovirus agents, methapyriline; chlorpheniramine, pyrilamine pheniramine, hydrocortisone, dexamethasone, fluocinolone, prednisone, prednisolone, methylprednisolone, fluorometholone, betamethasone, triamcinolone, phenylephrine, naphazoline, tetrahydrozoline, pilocarpine, carbachol, diisopropylfluorophosphate, echothiophate iodide, demecarium bromide, atropine sulfate, cyclopentolate, homatropine, scopolamine, tropicamide, eucatropine, epinephrine, heparin, antifibrinogen, fibrinolysin, anti clotting activase, acetohexamide, chlorpropamide, glipizide, glyburide, tolazamide, tolbutamide, insulin, aldose reductase inhibitors, thalidomide, 5-fluorouracil, adriamycin, asparaginase, azacytidine, azathioprine, bleomycin, busulfan, carboplatin, carmustine, chlorambucil, cisplatin, cyclophosphamide, cyclosporine, cytarabine, dacarbazine, dactinomycin, daunorubicin, estramustine, etoposide, etretinate, filgrastim, floxuridine, fludarabine, fluoxymesterone, flutamide, goserelin, hydroxyurea, ifosfamide, leuprolide, levamisole, lomustine, nitrogen mustard, melphalan, mercaptopurine, methotrexate, mitomycin, mitotane, pentostatin, pipobroman, plicamycin, procarbazine, sargramostim, streptozocin, tamoxifen, taxol, teniposide, thioguanine, uracil mustard, vinblastine, vindesine, pituitary hormones, insulin-related growth factor, thyroid hormones, growth hormones, heat shock proteins, immunological response modifiers such as muramyl dipeptide, interferons (including α, β, and γ interferons), interleukin-2, cytokines, FK506, tumor necrosis factor, thymopentin, transforming factor beta2, erythropoietin; antineogenesis proteins, monoclonal antibodies, brain nerve growth factor (BNGF), celiary nerve growth factor (CNGF), vascular endothelial growth factor (VEGF), monoclonal antibodies or aptamers directed against growth factors, and combinations and prodrugs thereof.

A therapeutic agent may be present in any suitable formulation for delivery to the eye. Methods well known in the art for making formulations are found, for example, in Remington: The Science and Practice of Pharmacy (20th ed., A. R. Gennaro ed., Lippincott: Philadelphia, 2000). Therapeutic agents may be administered to humans, domestic pets, livestock, or other animals with a pharmaceutically acceptable diluent, carrier, or excipient.

Therapeutic formulations may be liquid solutions, suspensions, or other formulations deliverable via a needle. Formulations may, for example, contain excipients, sterile water, saline, polyalkylene glycols such as polyethylene glycol, oils of vegetable origin, or hydrogenated napthalenes.

The therapeutic agent may be admixed with a pharmaceutically acceptable carrier adapted to provide sustained release of the therapeutic agent. Sustained release carriers include emulsions, suspensions, polymeric matrices, microspheres, microcapsules, microparticles, liposomes, multivesicular liposomes, lipospheres, hydrogels, salts, and polymers with the therapeutic agent reversibly bound electrostatically, chemically or by entrapment. Suitable sustained release formulations which may be injected into the therapeutic agent chamber are known in the art and are disclosed in, for example, U.S. Pat. Nos. 4,865,846, 4,115,544, 5,185,152, 4,078,052, 4,241,046, 4,853,224, 4,865,846, 6,309,669, 5,326,761, 6,071,534, 6,132,766 and 6,277,413 and PCTs WO 01/74400, WO 03/24420, WO 03/028765, WO 02/15888, and WO 03/070219, all of which are hereby incorporated in their entirety by reference.

Formulations of the drug may also include a transscleral diffusion promoting agent, such as dimethylsulfoxide, ethanol, dimethylformamide, propylene glycol, N-methylpyrolidone, oleic acid, isopropyl myristate, polar aprotic solvents, polar protic solvents, steroids, sugars, polymers, small molecules, charged small molecules, lipids, peptides, proteins, and surfactants.

A therapeutic agent may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid, phosphoric acid, or the like. Metal complexes include cations, such as divalent cations including calcium and magnesium, zinc, iron, and the like. In addition, a therapeutic agent may be optionally administered as a pharmaceutically acceptable pro-drug, e.g., an ester or amide..

The chemical compounds for use in such therapies may be produced and isolated as described herein or by any standard technique known to those in the field of medicinal chemistry. Conventional pharmaceutical practice may be employed to provide suitable formulations or compositions to administer the identified compound to patients suffering from a disease, disorder, or condition of the eye. Administration may begin before, during, or after the patient is symptomatic.

### Other Embodiments

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

All publications, patents, and patent applications mentioned in this specification are hereby incorporated by reference to the same extent as if each individual publication, patent, or patent application was specifically and individually to be incorporated by reference.

Other embodiments are in the claims.

What is claimed is:

1. A device for delivery of a therapeutic agent to a treatment site comprising:
an outer layer adapted for placement of the device at an internal body site; a reservoir within said outer layer, said reservoir adapted to form a closed system when containing a therapeutic agent;
a pressure equalization element adapted to maintain a substantially constant pressure within said closed system.

2. The device of claim 1 wherein the pressure equalization element comprises a membrane disposed within the outer layer, wherein the membrane divides the interior of the outer layer into a pressure equalizing chamber and a therapeutic agent chamber, wherein the volume of the pressure equalizing chamber changes in response to the volume in the therapeutic agent chamber in order to maintain a substantially constant pressure within the volume of the outer layer.

3. The device of claim 1 wherein the pressure equalization element comprises a channel.

4. The device of claim 2, further comprising a rate controlling membrane disposed between the membrane and the internal body surface.

5. The device of claim 1 wherein the outer layer further comprises an attachment element adapted for fixing said outer layer to an internal body surface.

6. The device of claim 5 wherein the attachment element comprises a rim.

7. The device of claim 6 further comprising a base that is contiguous with the rim and that contacts the internal body site and that has one or more openings to allow a therapeutic agent contained within the reservoir to contact the body site.

8. The device of claim 2, wherein the membrane is flexible.

9. The device of claim 8, wherein the membrane comprises silicone, polyvinyl alcohol, ethylene vinyl acetate, polylactic acid, nylon, polypropylene, polycarbonate, cellulose, cellulose acetate, polyglycolic acid, polylactic glycolic acid, a cellulose ester, polyethersulfone, an acrylic, polytetrafluoroethylene, polyfluorinated ethylenepropylenesilastic, Dacron, Mylar, ionic salts of alginate, polycaprolactone, urethanes, polyethylene, polymethylmethacrylate, a polyester, or mixtures thereof.

10. The device of claim 2, further comprising a port through which a therapeutic agent can be injected into the therapeutic agent chamber.

11. The device of claim 10, wherein the port comprises first and second septa, wherein the first septum separates the exterior of the device from the pressure equalizing chamber, and wherein the second septum separates the pressure equalizing chamber from the therapeutic agent chamber.

12. The device of claim 11, wherein the first and second septa are disposed such that a needle is capable of piercing both septa simultaneously.

13. The device of claim 10, wherein the port comprises a valve.

14. The device of claim 11, wherein the septa comprise a resealable material through which the therapeutic agent can be injected.

15. The device of claim 14, wherein the material is selected from the group consisting of silicone, polyvinyl alcohol, ethylene vinyl acetate, cellulose, cellulose acetate, a cellulose ester, polyethersulfone, polytetrafluoroethylene, polyfluorinated ethylenepropylenesilastic, Dacron, Mylar, polycaprolactone, urethanes, polyethylene, polymethylmethacrylate, a polyester, and mixtures thereof.

16. The device of claim 1, wherein the outer layer is comprised of a biocompatible, non-bioerodable material.

17. The device of claim 16, wherein the biocompatible, non-bioerodable material is selected from the group consisting of titanium, polypropylene, polyethylene, polycarbonate, polystyrene, polyester, urethane, nickel, nitinol, gold, tantalum, carbon, epoxy, and stainless steel.

18. The device of claim 1, further comprising a therapeutic agent disposed in the reservoir.

19. The device of claim 18, wherein the therapeutic agent is selected from the group consisting of small molecules, hormones, proteins, peptides, aptamers, lipids, DNA, RNA, PNA, enzymes, sugars, glycoproteins, polymers, metalloprotease, transition metals, antibodies, chelators, and combinations and prodrugs thereof.

20. The device of claim 19, wherein the therapeutic agent is an aptamer or prodrug thereof.

21. The device of claim 20, wherein the therapeutic agent is selected from the group consisting of anti-infectives; analgesics; antiallergenic agents; mast cell stabilizers; steroidal and non-steroidal anti-inflammatory agents; decongestants; anti-glaucoma agents; antioxidants; nutritional supplements; angiogenesis inhibitors; antimetabolites; fibrinolytics; wound modulating agents; neuroprotective drugs; angiostatic steroids; mydriatics; cyclopegic mydriatics; miotics; vasoconstrictors; vasodilators; anticlotting agents; anticancer agents; immunomodulatory agents; VEGF antagonists; immunosuppresant agents; and combinations and prodrugs thereof.

22. The device of claim 18, wherein the therapeutic agent is selected from the group consisting of 4,9(11)-pregnadien-17α,21-diol-3,20-dione, 4,9(11)-pregnadien-17α,21-diol-3,20-dione-21-acetate, timolol, betaxolol, atenolol, brimonidine, acetazolamide, methazolamide, dichlorphenamide, diamox, nimodipine, eliprodil, colchicine, vincristine, cytochalasin B, tetracycline, chlortetracycline, bacitracin, neomycin, polymyxin, gramicidin, oxytetracycline, chloramphenicol, gentamycin, erythromycin, sulfonamides, sulfacetamide, sulfamethizole, sulfisoxazole, fluconazole, nitrofurazone, amphotericin B, ketoconazole, trifluorothymidine, acyclovir, ganciclovir, didanosine, AZT, foscamet, vidarabine, idoxuridine, ribavirin, protease inhibitors, anti-cytomegalovirus agents, methapyriline; chlorpheniramine, pyrilamine pheniramine, hydrocortisone, dexamethasone, fluocinolone, prednisone, prednisolone, methylprednisolone, fluorometholone, betamethasone, triamcinolone, phenylephrine, naphazoline, tetrahydrozoline, pilocarpine, carbachol, diisopropylfluorophosphate, echothiophate iodide, demecarium bromide, atropine sulfate, cyclopentolate, homatropine, scopolamine, tropicamide, eucatropine, epinephrine, heparin, antifibrinogen, fibrinolysin, anti clotting activase, acetohexamide, chlorpropamide, glipizide, glyburide, tolazamide, tolbutamide, insulin, aldose reductase inhibitors, thalidomide, 5-fluorouracil, adriamycin, asparaginase, azacytidine, azathioprine, bleomycin, busulfan, carboplatin, carmustine, chlorambucil, cisplatin, cyclophosphamide, cyclosporine, cytarabine, dacarbazine, dactinomycin, daunorubicin, estramustine, etoposide, etretinate, filgrastim, floxuridine, fludarabine, fluoxymesterone, flutamide, goserelin, hydroxyurea, ifosfamide, leuprolide, levamisole, lomustine, nitrogen mustard, melphalan, mercaptopurine, methotrexate, mitomycin, mitotane, pentostatin, pipobroman, plicamycin, procarbazine, sargramostim, streptozocin, tamoxifen, taxol, teniposide, thioguanine, uracil mustard, vinblastine, vindesine, pituitary hormones, insulin, insulin-related growth factor, thyroid hormones, growth hormones, heat shock proteins, muramyl dipeptide, interferons, interleukin-2, cytokines, FK506, tumor necrosis factor, thymopentin, transforming factor beta2, erythropoietin; antineogenesis proteins, monoclonal antibodies, brain nerve growth factor (BNGF), celiary nerve growth factor (CNGF), vascular endothelial growth factor (VEGF), monoclonal antibodies or aptamers directed against growth factors, and combinations and prodrugs thereof.

23. The device of claim 18, wherein the therapeutic agent is pegaptanib sodium.

24. The device of claim 1 further comprising a catheter having a first end in fluid communication with the reservoir and a second end adapted for placement at an administration site.

25. The device of claim 5, wherein the device is affixed to the sclera.

26. The device of claim 6 wherein the device is affixed to the sclera throughout the circumference of the rim.

27. A syringe for injecting fluid into or withdrawing fluid from a closed system, the syringe comprising:
a) a barrel having a fluid portal end and a pressure generating end;
b) a needle having a hollow bore and being connected to the fluid portal end;
c) a venting tube having a hollow bore and being connected to the needle, wherein the hollow bores of the venting tube and the needle are not in fluid communication; and
d) a pressure source connected to the pressure generating end of the barrel.

28. The syringe of claim 27, wherein the hollow bores of the needle and the venting are disposed coaxial to one another.

29. The syringe of claim 27, furthering comprising:
e) a vacuum source in fluid communication with the needle.

30. The syringe of claim 27, further comprising:
e) a second needle having a hollow bore that is not in fluid communication with the hollow bore of the needle; and
f) a vacuum source in fluid communication with the hollow bore of the second needle.

31. The syringe of claim 30, wherein the hollow bores of the needle and the second needle are coaxial to one another.

32. The syringe of 31, wherein the hollow bores of the needle, the second needle, and the venting tube are coaxial to one another.

33. The syringe of claim 29, wherein the vacuum source is an evacuated canister.

34. The syringe of claim 33, further comprising a window for viewing the interior of the canister.

35. The syringe of claim 33, wherein the evacuated canister is removable and sealable.

36. The syringe of claim 33, wherein the evacuated canister further comprises a flexible diaphragm.

37. The syringe of claim 28, wherein the pressure source is a plunger.

38. A method of injecting fluid into or withdrawing fluid from a closed system, the method comprising the steps of:
a) providing a syringe of claim 27;
b) passing the needle and venting tube through a port into the system; and
c) injecting fluid into or withdrawing fluid from the system, wherein when fluid from the barrel is injected into the system through the needle, fluid inside the system exits through the venting tube in order to maintain a substantially constant pressure within the system, and when fluid from the system is pulled into the barrel through the needle, fluid outside the system enters the system through the venting tube in order to maintain a substantially constant pressure within the system.

39. The method of 38, wherein the closed system is a device comprising:
i) an outer layer adapted for placement on an internal body tissue so as to form a reservoir between said outer layer and said body tissue;
ii) a membrane disposed within the outer layer, wherein the membrane divides the interior of the outer layer into a pressure equalizing chamber and a therapeutic agent chamber, wherein the volume of the pressure equalizing chamber changes in response to the volume in the therapeutic agent chamber in order to maintain a substantially constant pressure within the volume of the outer layer; and
iii) an attachment element adapted for fixing said outer layer to an internal body surface that, when so affixed, defines a region which is in fluid communication with the therapeutic agent chamber;
wherein, in step (b), the needle is in fluid communication with the therapeutic agent chamber and the venting tube is in fluid communication with the pressure equalizing chamber; and
wherein when fluid from the barrel is injected into the therapeutic agent chamber through the needle, fluid inside the pressure equalizing chamber exits through the venting tube in order to maintain a substantially constant pressure within the device, and when fluid from the therapeutic agent chamber is pulled into the barrel through the needle, fluid outside the device enters the pressure equalizing chamber through the venting tube in order to maintain a substantially constant pressure within the device.

40. A method of injecting fluid into or withdrawing fluid from a closed system, the method comprising the steps of:
a) providing a syringe of claim 30;
b) passing the needle, the second needle, and the venting tube through a port into the system;
c) injecting fluid into or withdrawing fluid from the system;
wherein when fluid from the barrel is injected into the system through the needle, fluid inside the system exits through the venting tube in order to maintain a substantially constant pressure within the system, and when fluid in the system is pulled into the vacuum source through the second needle, fluid from outside the system enters the system through the venting tube in order to maintain a substantially constant pressure within the system.

41. The method of 40, wherein the closed system is a device comprising:
i) an outer layer adapted for placement on an internal body tissue so as to form a reservoir between said outer layer and said body tissue;
ii) a membrane disposed within the outer layer, wherein the membrane divides the interior of the outer layer into a pressure equalizing chamber and a therapeutic agent chamber, wherein the volume of the pressure equalizing chamber changes in response to the volume in the therapeutic agent chamber in order to maintain a substantially constant pressure within the volume of the outer layer; and
iii) an attachment element adapted for fixing said outer layer to an internal body surface that, when so affixed, defines a region which is in fluid communication with the therapeutic agent chamber;
wherein, in step (b), the needle and the second needle are in fluid communication with the therapeutic agent chamber and the venting tube is in fluid communication with the pressure equalizing chamber; and
wherein when fluid from the barrel is injected into the therapeutic agent chamber through the needle, fluid inside the pressure equalizing chamber exits through the venting tube in order to maintain a substantially constant pressure within the device, and when fluid in the therapeutic agent chamber is pulled into the vacuum source through the second needle, fluid from outside the device enters the pressure equalizing chamber through the venting tube in order to maintain a substantially constant pressure within the device.

42. The method of claim 41, wherein step (c) comprises the steps of:
i) actuating the vacuum source to remove fluid from the therapeutic agent chamber; and
ii) increasing the pressure in the barrel to inject fluid into the therapeutic agent chamber.

43. The method of claim 38, wherein the fluid injected into the therapeutic agent chamber comprises a therapeutic agent.

44. The method of claim 43, wherein the therapeutic agent is selected from the group consisting small molecules, hormones, proteins, peptides, aptamers, lipids, DNA, RNA, PNA, enzymes, sugars, glycoproteins, polymers, metalloprotease, transition metals, antibodies, chelators, and combinations and prodrugs thereof.

45. The method of claim 43, wherein the therapeutic agent is an aptamer or prodrug thereof.

46. The method of claim 43, wherein the therapeutic agent is selected from the group consisting of anti-infectives; analgesics; antiallergenic agents; mast cell stabilizers; steroidal and non-steroidal anti-inflammatory agents; decongestants; anti-glaucoma agents; antioxidants; nutritional supplements; angiogenesis inhibitors; antimetabolites; fibrinolytics; wound modulating agents; neuroprotective drugs; angiostatic steroids; mydriatics; cyclopegic mydriatics; miotics; vasoconstrictors; vasodilators; anticlotting agents; anticancer agents; immunomodulatory agents; VEGF antagonists; immunosuppresant agents; and combinations and prodrugs thereof.

47. The method of claim 43, wherein the therapeutic agent is selected from the group consisting of 4,9(11)-pregnadien-17α,21-diol-3,20-dione, 4,9(11)-pregnadien-17α,21-diol-3,20-dione-21-acetate, timolol, betaxolol, atenolol, brimonidine, acetazolamide, methazolamide, dichlorphenamide, diamox, nimodipine, eliprodil, colchicine, vincristine, cytochalasin B, tetracycline, chlortetracycline, bacitracin, neomycin, polymyxin, gramicidin, oxytetracycline, chloramphenicol, gentamycin, erythromycin, sulfonamides, sulfacetamide, sulfamethizole, sulfisoxazole, fluconazole, nitrofurazone, amphotericin B, ketoconazole, trifluorothymidine, acyclovir, ganciclovir, didanosine, AZT, foscamet, vidarabine, idoxuridine, ribavirin, protease inhibitors, anti-cytomegalovirus agents, methapyriline; chlorpheniramine, pyrilamine pheniramine, hydrocortisone, dexamethasone, fluocinolone, prednisone, prednisolone, methylprednisolone, fluorometholone, betamethasone, triamcinolone, phenylephrine, naphazoline, tetrahydrozoline, pilocarpine, carbachol, diisopropylfluorophosphate, echothiophate iodide, demecarium bromide, atropine sulfate, cyclopentolate, homatropine, scopolamine, tropicamide, eucatropine, epinephrine, heparin, antifibrinogen, fibrinolysin, anti clotting activase, acetohexamide, chlorpropamide, glipizide, glyburide, tolazamide, tolbutamide, insulin, aldose reductase inhibitors, thalidomide, 5-fluorouracil, adriamycin, asparaginase, azacytidine, azathioprine, bleomycin, busulfan, carboplatin, carmustine, chlorambucil, cisplatin, cyclophosphamide, cyclosporine, cytarabine, dacarbazine, dactinomycin, daunorubicin, estramustine, etoposide, etretinate, filgrastim, floxuridine, fludarabine, fluoxymesterone, flutamide, goserelin, hydroxyurea, ifosfamide, leuprolide, levamisole, lomustine, nitrogen mustard, melphalan, mercaptopurine, methotrexate, mitomycin, mitotane, pentostatin, pipobroman, plicamycin, procarbazine, sargramostim, streptozocin, tamoxifen, taxol, teniposide, thioguanine, uracil mustard, vinblastine, vindesine, pituitary hormones, insulin, insulin-related growth factor, thyroid hormones, growth hormones, heat shock proteins, muramyl dipeptide, interferons, interleukin-2, cytokines, FK506, tumor necrosis factor, thymopentin, transforming factor beta2, erythropoietin; antineogenesis proteins, monoclonal antibodies, brain nerve growth factor (BNGF), celiary nerve growth factor (CNGF), vascular endothelial growth factor (VEGF), monoclonal antibodies or aptamers directed against growth factors, and combinations and prodrugs thereof.

48. The method of claim 43, wherein the therapeutic agent is pegaptanib sodium.

49. A method of treating an ocular disease state in a mammal, the method comprising the steps of:
a) affixing to the sclera of the eye of the mammal the device of claim 1, and
b) loading the device with a therapeutic agent that treats the disease state.

50. The method of claim 49, wherein the therapeutic agent is selected from the group consisting of small molecules, hormones, proteins, peptides, aptamers, lipids, DNA, RNA, PNA, enzymes, sugars, glycoproteins, polymers, metalloprotease, transition metals, antibodies, chelators, and combinations and prodrugs thereof.

51. The method of claim 49, wherein the therapeutic agent is an aptamer or prodrug thereof.

52. The method of claim 49, wherein the therapeutic agent is selected from the group consisting of anti-infectives; analgesics; antiallergenic agents; mast cell stabilizers; steroidal and non-steroidal anti-inflammatory agents; decongestants; anti-glaucoma agents; antioxidants; nutritional supplements; angiogenesis inhibitors; antimetabolites; fibrinolytics; wound modulating agents; neuroprotective drugs; angiostatic steroids; mydriatics; cyclopegic mydriatics; miotics: vasoconstrictors; vasodilators; anticlotting agents; anticancer agents; immunomodulatory agents; VEGF antagonists; immunosuppresant agents; and combinations and prodrugs thereof.

53. The method of claim 49, wherein the therapeutic agent is selected from the group consisting of 4,9(11)-pregnadien-17α,21-diol-3,20-dione, 4,9(11)-pregnadien-17α,21-diol-3,20-dione-21-acetate, timolol, betaxolol, atenolol, brimonidine, acetazolamide, methazolamide, dichlorphenamide, diamox, nimodipine, eliprodil, colchicine, vincristine, cytochalasin B, tetracycline, chlortetracycline, bacitracin, neomycin, polymyxin, gramicidin, oxytetracycline, chloramphenicol, gentamycin, erythromycin, sulfonamides, sulfacetamide, sulfamethizole, sulfisoxazole, fluconazole, nitrofurazone, amphotericin B, ketoconazole, trifluorothymidine, acyclovir, ganciclovir, didanosine, AZT, foscamet, vidarabine, idoxuridine, ribavirin, protease inhibitors, anti-cytomegalovirus agents, methapyriline; chlorpheniramine, pyrilamine pheniramine, hydrocortisone, dexamethasone, fluocinolone, prednisone, prednisolone, methylprednisolone, fluorometholone, betamethasone, triamcinolone, phenylephrine, naphazoline, tetrahydrozoline, pilocarpine, carbachol, diisopropylfluorophosphate, echothiophate iodide, demecarium bromide, atropine sulfate, cyclopentolate, homatropine, scopolamine, tropicamide, eucatropine, epinephrine, heparin, antifibrinogen, fibrinolysin, anti clotting activase, acetohexamide, chlorpropamide, glipizide, glyburide, tolazamide, tolbutamide, insulin, aldose reductase inhibitors, thalidomide, 5-fluorouracil, adriamycin, asparaginase, azacytidine, azathioprine, bleomycin, busulfan, carboplatin, carmustine, chlorambucil, cisplatin, cyclophosphamide, cyclosporine, cytarabine, dacarbazine, dactinomycin, daunorubicin, estramustine, etoposide, etretinate, filgrastim, floxuridine, fludarabine, fluoxymesterone, flutamide, goserelin, hydroxyurea, ifosfamide, leuprolide, levamisole, lomustine, nitrogen mustard, melphalan, mercaptopurine, methotrexate, mitomycin, mitotane, pentostatin, pipobroman, plicamycin, procarbazine, sargramostim, streptozocin, tamoxifen, taxol, teniposide, thioguanine, uracil mustard, vinblastine, vindesine, pituitary hormones, insulin, insulin-related growth factor, thyroid hormones, growth hormones, heat shock proteins, muramyl dipeptide, interferons, interleukin-2, cytokines, FK506, tumor necrosis factor, thymopentin, transforming factor beta2, erythropoietin; antineogenesis proteins, monoclonal antibodies, brain nerve growth factor (BNGF), celiary nerve growth factor (CNGF), vascular endothelial growth factor (VEGF), monoclonal antibodies or aptamers directed against growth factors, and combinations and prodrugs thereof.

54. The method of claim 49, wherein the therapeutic agent is pegaptanib sodium.

55. The method of claim 49, wherein the therapeutic agent is admixed with a pharmaceutically acceptable carrier adapted to provide sustained release of the therapeutic agent.

56. The method of claim 55, wherein the carrier is selected from the group consisting of emulsions, suspensions, polymeric matrices, microspheres, microcapsules, microparticles, liposomes, multivesicular liposomes, lipospheres, hydrogels, salts, and polymers with the therapeutic agent reversibly bound electrostatically, chemically or by entrapment.

57. The method of claim 56, wherein the pharmaceutically acceptable carrier comprises a transscleral diffusion promoting agent.

58. The method of claim 57, wherein the promoting agent is selected from the group consisting of dimethylsulfoxide, ethanol, dimethylformamide, propylene glycol, N-methylpyrolidone, oleic acid, isopropyl myristate, polar aprotic solvents, polar protic solvents, steroids, sugars, polymers, small molecules, charged small molecules, lipids, peptides, proteins, and surfactants.

59. The method of claim 49, wherein the affixing comprises suturing, gluing, or sealing by means of one or more polymerizable compounds.

60. The method of claim 46, wherein the affixing comprises utilizing a biological healing mechanism.

61. The method of claim 60, wherein the biological healing mechanism is a postoperative adhesion, fibrotic encapsulation, or other foreign body reaction.

62. The method of claim 49, wherein step (b) is carried out two or more times with the same device.

63. The method of claim 49, wherein the device is affixed to the sclera over the equator of the eye.

64. The method of claim 49, wherein the device is affixed to the sclera over the pars planar of the eye.

65. The method of claim 49, wherein the disease state is macular degeneration, diabetic retinopathy, glaucoma, optic disc neovascularization, iris neovascularization, retinal neovascularization, choroidal neovascularization, pannus, pterygium, macular edema, vascular retinopathy, retinal vein occlusion, histoplasmosis, ischemic retinal disease, retinal degeneration, uveitis, inflammatory diseases of the retina, keratitis, cytomegalovirus retinitis, an infection, conjunctivitis, cystoid macular edema, cancer, or proliferative vitreoretinopathy.

66. The method of claim 65, wherein the disease state is macular degeneration.

67. The method of claim 49, wherein the therapeutic agent is delivered to the choroid or retina.

68. The method of claim 56 wherein the therapeutic agent comprises pegaptanib sodium.
